(19) United States
(12) Patent Application Publication
(10) Pub. No.: US 2012/0136261 A1
(43) Pub. Date: May 31, 2012

(54) Systems and methods for calibrating physiological signals with multiple techniques

(75) Inventors: Rakesh Sethi, Vancouver (CA); Robert Stoughton, Boulder, CO (US); James N. Watson, Dunfermline (GB); Paul Stanley Addison, Edinburgh (GB); Shannon E. Campbell, Boulder, CO (US); Daniel Lisogurski, Boulder, CO (US); Edward M. McKenna, Boulder, CO (US)

(73) Assignee: Nellcor Puritan Bennett LLC, Boulder, CO (US)

(21) Appl. No.: 12/956,932

(22) Filed: Nov. 30, 2010

(51) Int. Cl. A61B 5/021

(52) U.S. Cl. 600/485

(57) ABSTRACT

Systems and methods are disclosed herein for calibrating the calculation of physiological parameters. Two or more calibration techniques may be used to determine a relationship between physiological measurements and a desired physiological parameter, such as a relationship between differential pulse transit time (DPTT) and blood pressure. Different calibration techniques may be used in a serial fashion, one after the other, or in a parallel fashion, with different weights accorded to each calibration technique. When physiological or other changes occur, the calibration data may be stored for later use and new calibration data may be generated.

### SUMMARY

Continuous non-invasive blood pressure (CNIBP) monitoring systems allow a patient's blood pressure to be tracked continuously, unlike standard occlusion cuff techniques, and without the hazards of invasive arterial lines. Some CNIBP monitoring systems use multiple pulse oximetry type sensors to measure photoplethysmograph (PPG) signals at multiple body sites on a patient. The resulting multiple PPG signals may be analyzed to estimate the patient's blood pressure. When the locations of two sensors are at different distances or along different arterial paths from the heart (e.g., at the finger and forehead), a differential pulse transit time (DPTT) may be determined. A DPTT may represent the difference in the arrival times of a portion of a pulse wave between the two locations, and may be determined based on identifying a corresponding fiducial point in each of the two PPG signals (e.g., a maximum, minimum, specified percent distance between minimum and maximum, inflection point, or notch). The measured DPTT may then be used to determine a patient blood pressure value via a linear or non-linear relationship or model.

The accuracy of a blood pressure determination based on a DPTT determination may depend on the accuracy of the blood pressure-DPTT model. Calibration techniques may be used to provide initial model parameter values. However, over time or as a result of physiological changes or medical interventions, patient parameters such as blood vessel compliance may change, and the initially-calibrated model parameter values may introduce significant error into the model. In this case, recalibration may be necessary to maintain the accuracy of the blood pressure-DPTT model. Moreover, models generated from the use of multiple, different calibration techniques may provide accuracy improvements over models generated from only a single calibration technique.

According to one aspect of this disclosure, techniques for calibrating blood pressure-DPTT measurements are provided. The blood pressure-DPTT measurements may be calibrated using an inter-patient calibration technique, where a single or a few calibration points may be used to generate a blood pressure-DPTT model based on empirically-determined constants. The blood pressure-DPTT measurements may also be calibrated using an intra-patient calibration technique, where multiple calibration points measured for a single patient may be used to generate a blood pressure-DPTT model. In an embodiment, gravity-based or respiration-based calibration techniques may be used, where DPTTs measured with the patient in different physical or respiratory conditions are used to determine a blood pressure-DPTT model. In an embodiment, two or more of these techniques may be combined serially and/or in parallel to create a combined model. The combined techniques may be weighted differently based on calibration technique parameters, collection time, and/or the availability of calibration techniques. Calibration data may be analyzed to determine if the data is consistent with the current blood pressure-DPTT model. If not, the calibration data may be discarded or used to determine that a patient parameter has changed. When a patient parameter has changed, the current model may be saved for later use and a new model may be developed.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 depicts an illustrative patient monitoring system in accordance with an embodiment;

FIG. 2 is a block diagram of an illustrative patient monitoring system coupled to a patient in accordance with an embodiment;

FIG. 3 is a flow diagram of illustrative steps involved in an inter-patient empirical data calibration technique in accordance with an embodiment;

FIG. 4 is a flow diagram of illustrative steps involved in an intra-patient empirical data calibration technique in accordance with an embodiment;

FIG. 5 is a flow diagram of illustrative steps involved in a gravity-based calibration technique in accordance with an embodiment;

FIG. 6 is a flow diagram of illustrative steps involved in a respiration-based calibration technique in accordance with an embodiment;

FIG. 7 is a flow diagram of illustrative steps involved in a serial calibration technique in accordance with an embodiment; and

FIG. 8 is a flow diagram of illustrative steps involved in a multi-calibration technique in accordance with an embodiment.

### DETAILED DESCRIPTION

Monitoring the physiological state of a subject, for example, by determining, estimating, and/or tracking one or more physiological parameters of the subject, may be of interest in a wide variety of medical and non-medical applications. Knowledge of a subject's physiological characteristics (e.g., through a determination of one or more physiological parameters such as blood pressure, oxygen saturation, and presence of specific heart conditions) can provide short- and long-term benefits to the subject, such as early detection and/or warning of potentially harmful conditions, diagnosis and treatment of illnesses, and/or guidance for preventative medicine.

Physiological parameters of a subject can be determined from a signal representative of a patient's cardiac activity. For example, a tonometry signal can be obtained from a subject using a pressure transducer that may be fastened to subject's wrist area (or other suitable location). Alternatively, a photoplethysmograph (PPG) signal can be obtained using a PPG sensor in the form of an optical sensor that is clipped or fastened to a digit, appendage (e.g., an ear), or other part of the subject, such as the forehead. The term “digit” typically refers herein to a toe or finger of a subject. Such a PPG sensor may be used to emit and detect light that is used in oximetry settings to determine the blood oxygen saturation of a subject. The techniques described herein can be applied in many applications, including oximetry, continuous non-invasive blood pressure (CNIBP) determination, and heart rate monitoring. These techniques may use a single sensor operating at a single wavelength, a single sensor operating at multiple wavelengths, or multiple sensors operating with any combination of sensors operating at single or multiple wavelengths. For example, in some embodiments, the techniques described herein are applied in heart rate and CNIBP settings with one or more sensors operating at a single wavelength of light. In another example, in some embodiments, the techniques described herein are applied in oxygen saturation monitoring settings with one or more sensors operating at two or more wavelengths of light.

Further, a second PPG sensor may be affixed to a subject, and the combination of these two PPG sensors may allow for the determination of the subject's blood pressure, for example, using continuous non-invasive blood pressure (CNIBP) techniques. For example, in an arrangement, two PPG-based oximetry sensors can be used. One of these sensors may be used to determine the blood oxygen saturation of the subject, and/or both sensors may be used in combination to determine an estimate of the blood pressure of the subject via non-invasive techniques.

In an arrangement, a PPG sensor may be affixed to a subject. As described above, this PPG sensor may correspond to a pulse oximetry sensor (and may be used as a single sensor to determine a blood oxygen saturation level, and/or as one of two sensors in tandem to determine a subject blood pressure). The PPG sensor may emit light that is passed through or reflected by the tissue of a subject and detected by a detector. The light passed through or reflected by the tissue may be selected to be of one or more wavelengths that are absorbed by the subject's blood in an amount representative of the amount of the blood constituent present in the blood. In some embodiments, multiple wavelengths are multiplexed using established methods. The amount of light passed through or reflected by the tissue varies in accordance with the changing amount of blood constituent in the tissue and the related light absorption. Red and infrared (IR) wavelengths may be used because it has been observed that highly oxygenated blood will absorb relatively less red light and more infrared light than blood with a lower oxygen saturation. In some embodiments, the oxygen saturation of blood can be determined by comparing the ratio of AC and DC components in the red signal to the ratio of AC and DC components in the infrared signal.

In an arrangement, at least two PPG sensors may be affixed to a subject. As described above, these PPG sensors may correspond to pulse oximetry sensors, and may be used to determine a CNIBP of a subject. Each sensor may be positioned at a different location on a subject's body to estimate the blood pressure and/or other related biosignal parameters of the subject from a measured signal or signals. In an arrangement, a first reference point of a first signal measured at the first sensor may be identified (and this reference point may correspond to a reference “feature,” such as a leading or trailing edge of the signal, or the location of a signal peak or valley), and a second reference point of a second signal measured at the second sensor may be identified. The first and second reference points need not correspond to the same type of reference “feature” (e.g., two valleys). Indeed, in some embodiments, the first and second reference points correspond to different signal features, each of which are chosen based on the types of features that are easiest to identify at their corresponding body locations.

Once the arrival times of the first and second reference points are identified, the elapsed time between the arrival times, denoted T, may be determined. An estimate of the subject's blood pressure, p, may then be determined from any suitable relationship between the blood pressure and T. For example, in an arrangement, the following mathematical relation may be used to determine an estimate of subject blood pressure from the elapsed time

p=a+b·ln(T),

where a and b are constants that may be determined from a calibration process and may be dependent on the nature of the subject and signal detector that is, for example, affixed to the subject. Once calibration has been completed, for example, using a non-invasive blood pressure device, an equation similar or identical to the one above can be used to determine a subject blood pressure. The equation above is meant to be illustrative, and any other suitable equation (or equations) may also e used to derive an estimated subject blood pressure. Further, blood pressure estimates may be computed on a continuous basis (e.g., once per heartbeat) or a periodic basis (e.g., at multi-heartbeat intervals).

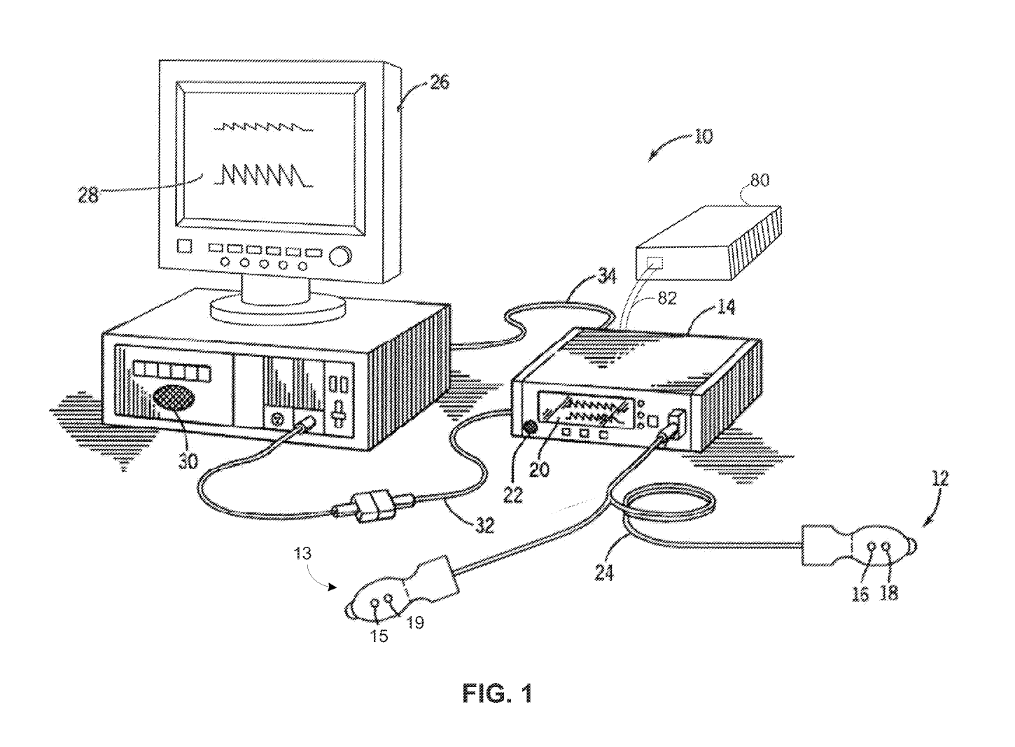

FIG. 1 shows an illustrative patient monitoring system 10. System 10 may include a sensor unit 12 and a monitor 14. In an embodiment, sensor unit 12 is part of a continuous, non-invasive blood pressure (CNIBP) monitoring system. In an embodiment, sensor unit 12 may include an emitter 16 for emitting light at one or more wavelengths into a patient's tissue. A detector 18 may also be provided in sensor unit 12 for detecting the light originally from emitter 16 that emanates from the patient's tissue after passing through the tissue. Any suitable physical configuration of emitter 16 and detector 18 may be used. In an embodiment, sensor unit 12 may include multiple emitters and/or detectors, which may be spaced apart. In an embodiment, system 10 may include one or more additional sensor units, such as sensor unit 13, which may take the form of any of the embodiments described herein with reference to sensor unit 12. For example, sensor unit 13 may include emitter 15 and detector 19. Sensor unit 13 may be the same type of sensor unit as sensor unit 12, or sensor unit 13 may be of a different sensor unit type than sensor unit 12. Sensor units 12 and 13 may be capable of being positioned at two different locations on a subject's body; for example, sensor unit 12 may be positioned on a patient's forehead, while sensor unit 13 may be positioned at a patient's fingertip. As discussed in additional detail below, one or more signals from one or more sensors and/or sensor units may be used in the techniques described herein.

Sensor units 12 and 13 may each detect any signal that carries information about a patient's physiological state, such as the pulsatile force exerted on the walls of an artery using, for example, oscillometric methods with a piezoelectric transducer and an occlusion device, such as a cuff (not shown). According to another embodiment, system 10 may include a plurality of sensors forming a sensor array in lieu of either or both of sensor units 12 and 13. It will be understood that any type of sensor, including any type of physiological sensor, may be used in one or more of sensor units 12 and 13 in accordance with the systems and techniques disclosed herein. It is understood that any number of sensors measuring any number of physiological signals may be used to assess patient status in accordance with the techniques described herein.

According to an embodiment, system 10 may include a plurality of sensors forming a sensor array in lieu of a single sensor, for example, sensor unit 12. Each of the sensors of the sensor array may be a complementary metal oxide semiconductor (CMOS) sensor. Alternatively, each sensor of the array may be charged coupled device (CCD) sensor. In another embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

In some embodiments, the signal obtained from a sensor or probe, such as sensor units 12 or 13, may take the form of a PPG signal obtained, for example, from a CNIBP monitoring system or pulse oximeter. In this embodiment, sensor units 12 and 13 may each include a light sensor that is placed at a site on a patient, typically a fingertip, toe, forehead or earlobe, or in the ease of a neonate, across a foot. The system may pass light using a light source through blood perfused tissue and photoelectrically sense the absorption of light in the tissue. For example, the system may measure the intensity of light that is received at the light sensor as a function of time. The light intensity or the amount of light absorbed may then be used to calculate physiological measurements (e.g., blood pressure and blood oxygen saturation). Techniques for obtaining blood pressure measurements from data are described in more detail in co-pending, commonly assigned U.S. patent application Ser. No. 12/242,867, filed Sep. 30, 2008, entitled “SYSTEMS AND METHODS FOR NON-INVASIVE CONTINUOUS BLOOD PRESSURE DETERMINATION” and co-pending, commonly assigned U.S. patent application Ser. No. 12/242,238, filed Sep. 30, 2008, entitled “SYSTEMS AND METHODS FOR NON-INVASIVE BLOOD PRESSURE MONITORING,” which are both hereby incorporated by reference herein in their entireties.

It will be understood that the present disclosure is applicable to any suitable signals that communicate information about an underlying physiological process. It will be understood that the signals may be digital or analog. Moreover, it will be understood that the present disclosure has wide applicability to signals including, but not limited to other biosignals and combinations of biosignals. For example, the techniques of the present disclosure could be applied to monitoring pathological sounds or arterial (or venous) pressure fluctuations.

In an embodiment, sensor units 12 and 13 may be connected to and draw power from monitor 14 as shown. In another embodiment, sensor units 12 and 13 may be wirelessly connected to monitor 14 and include their own batteries or similar power supplies (not shown). In an embodiment, sensor units 12 and 13 may be communicatively coupled to monitor 14 via cables such as cable 24. However, in other embodiments, a wireless transmission device (not shown) or the like may be used instead of or in addition to cable 24.

Monitor 14 may be configured to calculate physiological parameters (e.g., heart rate, blood pressure, blood oxygen saturation) based at least in part on data received from one or more sensor units such as sensor units 12 and 13. In an alternative embodiment, the calculations may be performed on the monitoring device itself and the result of the calculations may be passed to monitor 14. Further, monitor 14 may include a display 20 configured to display the physiological parameters or other information about the system. In the embodiment shown, monitor 14 may also include a speaker 22 to provide an audible sound that may be used in various other embodiments to be discussed further below, such as for example, sounding an audible alarm in the event that a patient's physiological parameters are not within a predefined normal range. Monitor 14 may also include a measurement quality indicator, such as a graphic or text in display 20 or a tone or message via speaker 22.

In the illustrated embodiment, system 10 may also include a multi-parameter patient monitor 26. The monitor 26 may include a cathode ray tube display, a flat panel display (as shown) such as a liquid crystal display (LCD) or a plasma display, or may be any other type of monitor now known or later developed. Multi-parameter patient monitor 26 may be configured to calculate physiological parameters and to provide a display 28 for information from monitor 14 and from other medical monitoring devices or systems (not shown). For example, multi-parameter patient monitor 26 may be configured to display an estimate of a patient's blood pressure from monitor 14, blood oxygen saturation generated by monitor 14 (referred to as an “SpO₂” measurement), and pulse rate information from monitor 14. Monitor 26 may include a speaker 30.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown), or with each other in addition to or instead of cable 32 or 34. Monitor 14 may be powered by a battery (not shown) or by a conventional power source such as a wall outlet.

Calibration device 80, which may be powered by monitor 14 via a cable 82, a battery, or by a conventional power source such as a wall outlet, may include any suitable physiological signal calibration device. Calibration device 80 may be communicatively coupled to monitor 14 via cable 82, and/or may communicate wirelessly (not shown). For example, calibration device 80 may take the form of any invasive or non-invasive physiological monitoring or measuring system used to generate reference physiological measurements for use in calibrating a monitoring device. For example, calibration device 80 may take the form of a blood pressure monitoring system, and may include, for example, an aneroid or mercury sphygmomanometer and occluding cuff, a pressure sensor inserted directly into a suitable artery of a patient, an oscillometric device or any other device or mechanism used to sense, measure, determine, or derive a reference blood pressure measurement. In some embodiments, calibration device 80 may include a manual input device (not shown) used by an operator to manually input reference physiological measurements obtained from some other source (e.g., an external invasive or non-invasive physiological measurement system).

Calibration device 80 may also access reference measurements stored in memory (e.g., RAM, ROM, or a storage device). As described in more detail below, the reference measurements generated or accessed by calibration device 80 may be updated in real-time, resulting in a continuous source of reference measurements for use in continuous or periodic calibration. Alternatively, reference measurements generated or accessed by calibration device 80 may be updated periodically, and calibration may be performed on the same periodic cycle. In the depicted embodiment, calibration device 80 is connected to monitor 14 via cable 82. In other embodiments, calibration device 80 may be a stand-alone device that may be in wireless communication with monitor 14. Reference measurements may then be wirelessly transmitted to monitor 14 for use in calibration. In some embodiments (not shown), the calibration device 80 is part of the multi-parameter monitor 26 and monitor 14 may obtain calibration information through one of the links between the two systems (such as cable 32 or 34 or by a wireless link). In still other embodiments, calibration device 80 is completely integrated within monitor 14. For example, in some embodiments, calibration device 80 may access reference measurements from a relational database stored within calibration device 80, monitor 14, or multi-parameter patient monitor 26. As described in additional detail below, calibration device 80 may be responsive to a recalibration signal, which may initiate the calibration of monitor 14 or may communicate recalibration information to calibration device 80 (e.g., a recalibration schedule). Calibration may be performed at any suitable time (e.g., once initially after monitoring begins) or on any suitable schedule (e.g., a periodic or event-driven schedule). In an embodiment, calibration may be initiated or delayed based at least in part on a measurement quality assessment or a recalibration initiation assessment. Techniques for recalibrating a continuous, non-invasive blood pressure (CNIBP) system are described in more detail in co-pending, commonly assigned U.S. patent application Ser. No. 12/242,858, filed Sep. 30, 2008, entitled “SYSTEMS AND METHODS FOR RECALIBRATING A NON-INVASIVE BLOOD PRESSURE MONITOR,” which is hereby incorporated by reference herein in its entirety.

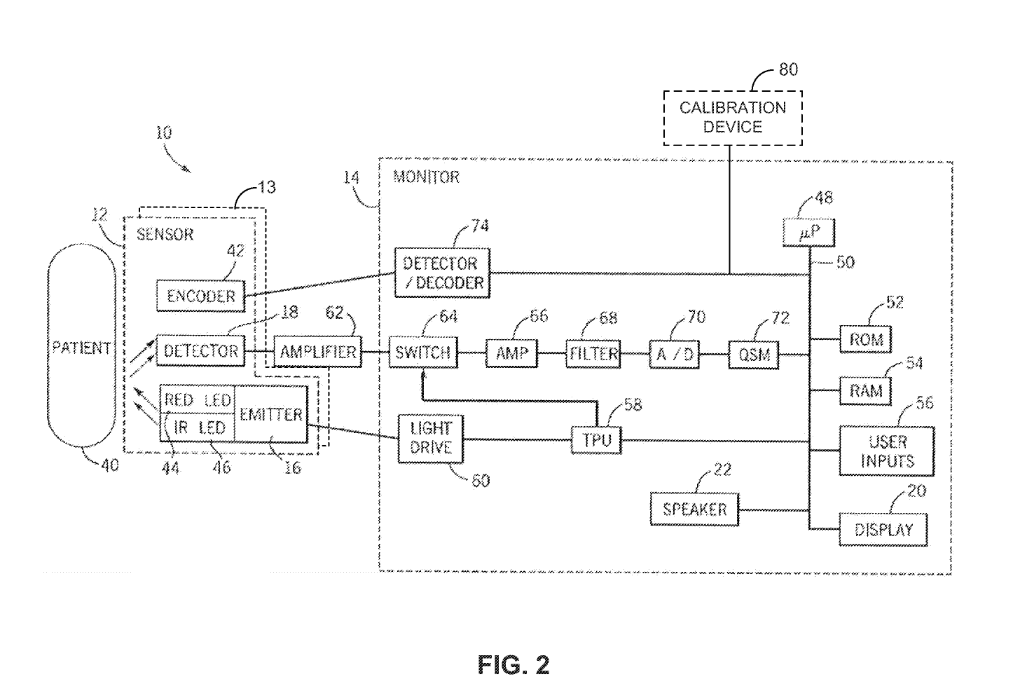

FIG. 2 is a block diagram of patient monitoring system 10 of FIG. 1, which may be coupled to a patient 40 in accordance with an embodiment. Certain illustrative components of sensor unit 12 and monitor 14 are illustrated in FIG. 2. Because sensor units 12 and 13 may include similar components and functionality, only sensor unit 12 will be discussed in detail for ease of illustration. It will be understood that any of the concepts, components, and operation discussed in connection with sensor unit 12 may be applied to sensor unit 13 as well (e.g., emitter 16 and detector 18 of sensor unit 12 may be similar to emitter 15 and detector 19 of sensor unit 13). It will be noted that patient monitoring system 10 may include one or more additional sensor units or probes, which may take the form of any of the embodiments described herein with reference to sensor units 12 and 13 (FIG. 1). These additional sensor units included in system 10 may take the same form as sensor unit 12, or may take a different form. In an embodiment, multiple sensors (distributed in one or more sensor units) may be located at multiple different body sites on a patient.

Sensor unit 12 may include encoder 42. In an embodiment, encoder 42 may contain information about sensor unit 12, such as what type of sensor(s) it includes (e.g., whether the sensor is a pressure transducer or a pulse oximeter). This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information about a patient's characteristics may allow monitor 14 to determine, for example, patient-specific threshold ranges in which the patient's physiological parameter measurements should fall and to enable or disable additional physiological parameter algorithms. This information may also be used to select and provide coefficients for equations from which, for example, blood pressure and other measurements may be determined based at least in part on the signal or signals received at sensor unit 12. For example, some pulse oximetry sensors rely on equations to relate an area under a pulse of a photoplethysmograph (PPG) signal to determine blood pressure. These equations may contain coefficients that depend upon a patient's physiological characteristics as stored in encoder 42. In some embodiments, encoder 42 may include a memory or a coded resistor which stores one or more of the following types of information for communication to monitor 14: the types of sensors included in sensor unit 12; the wavelength or wavelengths of light used by an oximetry sensor when included in sensor unit 12; a signal threshold for each sensor in the sensor array; any other suitable information; or any combination thereof. Encoder 42 may also include information about the recalibration requirements of the sensors included in sensor unit 12, including any one of a nominal frequency of recalibration and preferred recalibration conditions. The stored information, including recalibration frequency, may also be adjusted or further tuned through the user interface on monitor 14, or by a connected device through cables 32 or 34 of FIG. 1 or through a network or wireless connection (not shown).

In an embodiment, signals from sensor unit 12 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22. Microprocessor 48 may be a microcontroller or digital signal processing (DSP) chip using Flash or one-time programmable (OTP) memory. Monitor 14 may also include hardware accelerators (e.g., an ADI SHARC DSP or AMI Belasigna DSP) to implement one or more steps of the techniques described herein. In some embodiments, monitor 14 includes an FPGA or ASIC with one or more synthesized or dedicated hardware processor cores and memory, and/or dedicated hardware units such as multipliers, filters, or digital logic components.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but are not limited to, RAM, ROM, EPROM, EEPROM, one-time programmable (OTP) memory, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the red LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/D converter 70 for multiple light wavelengths or spectra received.

In an embodiment, system 10 may include a stimulus drive (not shown), which may control when a stimulus is used to apply a signal to the patient, the response to which communicates information about the patient's physiological processes. Techniques for obtaining physiological measurements by inducing perturbations in a patient via a stimulus drive are described in more detail in co-pending, commonly assigned U.S. patent application Ser. No. 12/248,738, filed Oct. 9, 2008, entitled “SYSTEMS AND METHODS USING INDUCED PERTURBATION TO DETERMINE PHYSIOLOGICAL PARAMETERS,” which is incorporated by reference herein in its entirety. It will be noted that embodiments of system 10 may include necessary control and drive circuitry suitable for the type of sensors included in sensor unit 12 (e.g., instead of or in addition to TPU 58 and/or light drive circuitry 60).

In an embodiment, microprocessor 48 may determine the patient's physiological parameters, such as blood pressure or blood oxygen saturation, using various algorithms and/or look-up tables based at least in part on the value of the received signals and/or data from sensor unit 12. For example, when sensor unit 12 includes an oximetry sensor, microprocessor 48 may generate an equation that represents empirical data associated with one or more patients that includes various blood pressure measurements associated with different areas under a pulse of a PPG signal. Signals corresponding to information about patient 40 may be transmitted from encoder 42 to a decoder 74. These signals may include, for example, encoded information relating to patient characteristics. Decoder 74 may translate these signals to enable the microprocessor to determine signal and/or patient-specific thresholds or threshold ranges based at least in part on algorithms or look-up tables stored in ROM 52 or other memory. In some embodiments, decoder 74 interfaces with an external bus or parallel or serial port. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56. Various embodiments may include or exclude any one or more of display 20, speaker 22 and user inputs 56. Additionally, any one or more steps of the techniques described herein may be distributed across one or more local or remote hardware components, for example, when processed values and user programmed parameters are transmitted via cable 32 or 34 or from a wireless interface (not shown). In some embodiments, the entire system is ambulatory and the results are stored in memory until the patient returns the unit so that information may be downloaded or a wireless link is activated to download the information or to stream it in real-time.

Patient monitoring system 10 may also include calibration device 80. Although shown external to monitor 14 in the example of FIGS. 1-2, calibration device 80 may additionally or alternatively be internal to monitor 14. Calibration device 80 may be connected to internal bus 50 of monitor 14, or an external bus configured to address external parallel devices (not shown). In some embodiments, calibration device 80 communicates over a serial interface such as a UART, asynchronous or synchronous serial port, SPI I2C, USB, Bluetooth, IEEE 802.15.4 or other wired or wireless interface. As described above, reference measurements from calibration device 80 may be accessed by microprocessor 48 for use in calibrating the sensor measurements and determining physiological signals from the sensor signal and empirical data of one or more patients.

As discussed above, the signal from the patient can be degraded by noise, among other sources. One source of noise is electromagnetic coupling from other electronic instruments. Movement of the patient also introduces noise and affects the signal. For example, the contact between the sensor and the skin can be temporarily disrupted when movement causes either to move away from the skin. Another source of noise is ambient light that reaches the light detector in an oximetry system.

Noise (e.g., from patient movement) can degrade a sensor signal relied upon by a care provider, without the care provider's awareness. This is especially true if the monitoring of the patient is remote, the motion is too small to be observed, or the care provider is watching the instrument or other parts of the patient, and not the sensor site. Processing sensor signals may involve operations that reduce the amount of noise present in the signals or otherwise identify noise components in order to prevent them from affecting measurements of physiological parameters derived from the sensor signals.

It will be understood that the present disclosure is applicable to any suitable signals and that physiological signals may be used merely for illustrative purposes. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

As described above, the following mathematical relation may be used to determine an estimate of subject blood pressure from the elapsed time between the arrival of a signal reference point or feature at two sensors with different locations:

p=a+b·ln(T),

where T is the elapsed time, also known as the differential pulse transit time (DPTT), and a and b are constants that may be determined for each individual subject by performing a calibration.

In an embodiment, one way to calibrate the system for a specific individual subject is to use a blood pressure measurement device such as a sphygmomanometer or an automated blood pressure device at two times when the blood pressure is different. This will yield two equations, which may then be used to solve for the constants a and b.

In another embodiment, the constants a and b may be solved for via a linear relationship, using an inter-patient empirical data calibration technique. Clinical trials have been conducted on a number of subjects to experimentally measure the parameters a and b, and it was determined that a and b are generally related linearly to each other by the equation

a=c₁+c₂*b,

where c₁ and c₂ are empirically determined constants. While c₁ and c₂ for a systolic measurement differ from c₁ and c₂ for a diastolic measurement, c₁ and c₂ for both the systolic and diastolic cases are generally constant across multiple subjects. Thus, since c₁ and c₂ are both empirically determined, and a and b are related to each other via c₁ and c₂, only one of a and b needs to be measured to determine the other parameter, and thus only one calibration point is needed to determine the relationship between the DPTT and the blood pressure of a patient.

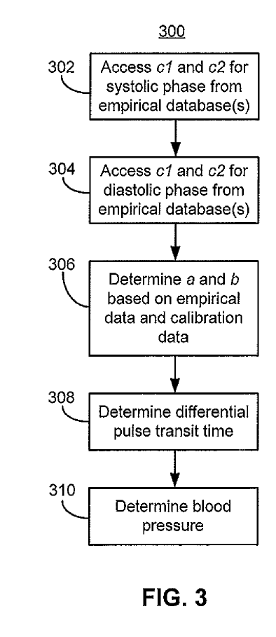

FIG. 3 is a flow diagram 300 of illustrative steps involved in a blood pressure measurement with an inter-patient empirical data calibration technique, which may be performed by, for example, calibration device 80 (FIGS. 1-2). The flow diagram begins at step 302, where empirically-determined c₁ and c₂ values for the systolic phase are accessed by, for example, calibration device 80 (FIGS. 1-2). The calibration device 80 may access the empirically-determined values of c₁ and/or c₂ from, for example, one or more internal database(s) in the calibration device 80. In some embodiments, the calibration device 80 may access the empirical c₁ and/or c₂ values from one or more external database(s). Optionally, an operator may manually input values for c₁ and/or c₂ obtained from some other source via a manual input device (not shown). The values c₁ and/or c₂ may also be obtained through any wired or wireless connection with a hospital information system or some other networked device with access to empirical or patient information.

At step 304, values of c₁ and c₂ for the diastolic phase are accessed, similar to how values for c₁ and c₂ were accessed in step 302. Values for a and b are then determined (step 306) based on the systolic and/or the diastolic c₁ and/or c₂ values and calibration data (e.g., a blood pressure reading taken from calibration device 80 and a corresponding DPTT). The patient's differential pulse transit time (DPTT) is measured (step 308) and the patient's blood pressure is determined (step 310) based on the measured DPTT, the determined values for a and b, and the relationship between DPTT and blood pressure discussed above. However, in other embodiments, other methods and techniques for relating DPTT and blood pressure may be used. The DPTT measurement and/or the patient blood pressure determination may be performed by, for example, sensor units 12 and 13 operating in conjunction with monitor 14, as described above in relation to FIGS. 1-2.

While the use of the single calibration point method to generate DPTT-blood pressure models is valid and works well, if blood pressure changes significantly (for example greater than 20-30% or 20-30 mm Hg from the calibration point), the accuracy of the model may decrease. Thus, the use of multiple calibration points for a single patient may provide a more accurate representation of the DPTT-blood pressure relationship. This relationship may be linear or non-linear, and may be extrapolated and/or interpolated to define the relationship beyond the range of the collected calibration points.

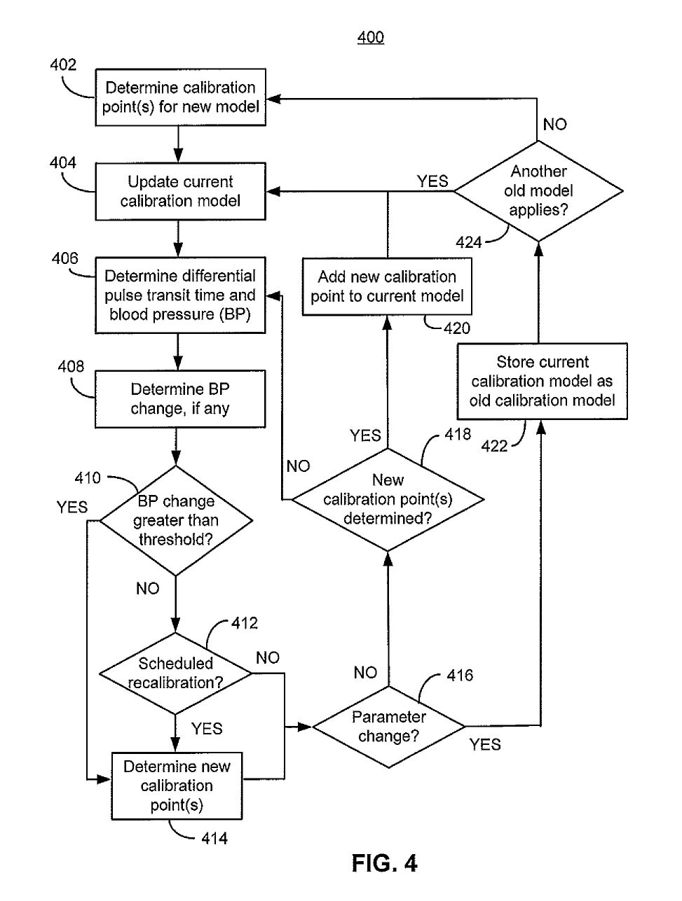

FIG. 4 is a flow diagram of illustrative steps involved in a multi-point intra-patient empirical data calibration technique 400, which may be performed by patient monitoring system 10 (described above in relation to FIGS. 1-2). At step 402, the monitoring system 10 first determines one or more calibration points relating blood pressure to DPTT. This may be performed by, for example, taking successive blood pressure cuff measurements, or by any other method of measuring the DPTT-blood pressure relationship. At step 404, the monitoring system 10 then either generates a current calibration model with the determined calibration points, or, if a current calibration model exists, updates the current calibration model with the determined calibration point. Next, the monitoring system 10 determines DPTT and the blood pressure according to the current calibration model (step 406). The monitoring system 10 may determine the DPTT measurement and blood pressure according to the techniques and embodiments described above. After determining the blood pressure, the monitoring system 10 may determine if the blood pressure has changed by, for example, comparing the most recent blood pressure measurement to one or more preceding blood pressure measurements (step 408). If the blood pressure has changed, the monitoring system 10 may determine if the blood pressure change exceeds a particular threshold (step 410). This threshold may be predetermined, or may be dynamically updated, for example, via manual entry by an operator or via automatic generation by the monitoring system 10 or an external source linked to the monitoring system 10.

If, at step 410, the monitoring system 10 detects that the blood pressure change has exceeded the threshold, it may initiate a recalibration process to determine one or more new calibration points for the current calibration model (step 414). Alternatively, if the monitoring system 10 determines that no blood pressure change has occurred, or any blood pressure change falls beneath the threshold, then the monitoring system 10 may check to see if a recalibration has been scheduled (step 412). Recalibrations may be scheduled at designated time intervals selected by the user, the operator, or dynamically determined by the monitoring system 10 or an external source coupled to the monitoring system 10. If the monitoring system 10 determines that a recalibration has been scheduled, then the monitoring system initiates a recalibration process to determine one or more new calibration points for the current calibration model (step 414).

If, at step 412, the monitoring system 10 determines that no recalibration has been scheduled, or if one or more new calibration points for the current calibration model have been determined at step 414, the monitoring system 10 may check to see if a change event has occurred. For example, the monitoring system 10 may check to see if any one or more of the following patient parameters have changed: compliance value, body position, height of patient's arm, posture including arm or wrist position, vessel diameter, oxygen saturation, respiration rate, effort of respiration, the presence of arrhythmia, wall stiffness, mean blood pressure, local blood pressures, pressure of sensor or bandage against skin, intra-thoracic pressure, ejection fraction, stroke volume, heart rate, inotropic effects, viscosity, blood volume and local skin temperature. A change event may affect the measurements taken with one or more sensors. The monitoring system 10 may determine if a change event has occurred by comparing a measured patient parameter value with a previously measured or stored value for the same parameter, which may be associated with the current calibration model. If the measured parameter value differs from the previously measured or stored value by a certain threshold, which may be dynamic or pre-determined, the monitoring system 10 may determine that a change event has occurred. For example, if one or more new calibration points determined in step 414 do not match with previous calibration points, or significantly deviate from the calibration model, the monitoring system 10 may decide that a change event has occurred. In some embodiments, the monitoring system 10 is configured to detect a change in a compliance value by identifying changes in one or more morphological parameters of a PPG signal, including augmentation index, pulse amplitude (which may be normalized), the area under a portion of a PPG waveform (or a normalized area), a time difference between the main peak and the dichrotic notch, the relative position of other fiducial points, rise time, the relationship between two or more consecutive pulse signals, or any combination thereof. Additional examples of changes that may be detected by the monitoring system 10 are described in Millasseau et al., “Determination of age-related increases in large artery stiffness by digital pulse contour analysis,” Clinical Science, v, 103, pp. 371-377, 2002, incorporated by reference herein in its entirety.

If the monitoring system 10 determines that no change event has occurred, the system may go to step 418 and check if one or more new calibration points for the current calibration model have been determined at step 414. If not, the monitoring system 10 may return to step 406 to determine the differential pulse transit time and blood pressure. If, instead, new calibration point(s) have been determined, the monitoring system 10 may add the new calibration points to the current calibration model (step 420) and proceed to update the current calibration model (step 404). The current calibration model may be updated by, for example, extrapolation or interpolation based on the new calibration point(s). Optionally, when there is more than one calibration point to consider, different weighting methods may be used to emphasize specific calibration points or groups of calibration points. For example, more recent calibration points may be weighted more heavily than older calibration points, thus emphasizing the more heavily-weighted recent points over the less heavily-weighted older points. As another example, calibration points or groups of calibration points closer to a “best fit” curve for the model or to the pressure-DPTT relationship curve could be weighted more heavily (and thus emphasized more) than calibration points farther away from the “best fit” curve or the pressure-DPTT relationship curve. During the calibration model update process, or at any other time, the calibration points in the current calibration model may be examined, and one or more calibration points may be discarded if it is determined that they are outliers. In some embodiments, the monitoring system 10 may initiate another recalibration to confirm that points deemed outliers are actually outliers before discarding them.

However, if the monitoring system 10 determines at step 416 that a change event has occurred, then it may proceed to determine a new calibration model in steps 422 and 424. In some embodiments, if a significant change to one or more patient parameters occurs, subsequent calibration points may not be incorporated into the current calibration model to provide an accurate DPTT-blood pressure relationship. Instead, these calibration points may be used in a new calibration model. However, if the patient parameter later changes back to the parameter value associated with the current calibration model, then the current calibration model can be used again. Therefore, upon detecting a change event, the monitoring system 10 first stores the current calibration model as an old calibration model at step 420. Thus, if the patient parameter later changes back to the parameter value associated with the now-old calibration model, the calibration data associated with the old calibration model can be reused.

Next, the monitoring system 10 checks to see if another old calibration model applies for the new patient parameter value (step 422). If an old calibration model is found that does apply for the new patient parameter value, then the monitoring system 10 may load that old calibration model and set it as the current calibration model. In this case, the monitoring system 10 may also take a calibration measurement and update the now-current calibration model. Optionally, if one or more new calibration points were determined at step 414, these new calibration point(s) may be used to update the now-current calibration model. If the monitoring system 10 does not find an old calibration model that applies, it proceeds to step 402, where it may determine one or more new calibration points for a new model, or, optionally, use new calibration point(s) determined at step 414, if any.

While in the description above, the steps of process 400 occur in a particular order, in other embodiments, the steps may be rearranged in any suitable order. For example, the monitoring system 10 may check for a scheduled recalibration (step 412) after it checks for a change event (step 416), or before it checks if blood pressure change exceeds a threshold (step 410).

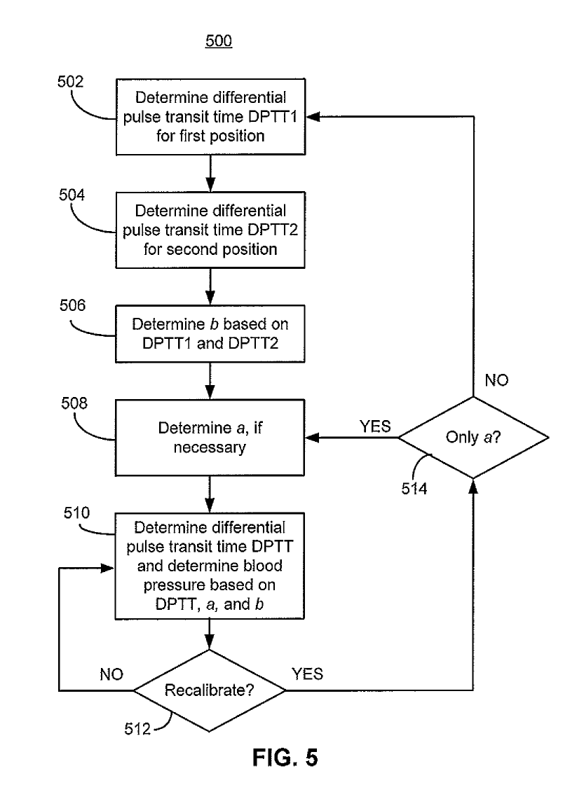

Another calibration technique is a gravity-based calibration technique, described below in more detail with regards to FIG. 5. Pulse transit times, differential and otherwise, depend at least in part on the pressure gradient along the vessel path for which the pulse transit times are being measured. If a known pressure gradient along the vessel path of interest can be induced, then the measured pulse transit time may be used to determine the relationship between pulse transit time and pressure. This known pressure gradient may be induced via gravity. For example, a first measurement of DPTT may be made with the patient in a reference configuration, such as a supine position. The patient's body may then be supported in a second configuration in which gravity creates a hydrostatic pressure difference in the vessel path relative to the first configuration, and a second DPTT measurement may be made. The ratio of the average hydrostatic pressure difference to the measured change in DPTT is then the rate at which pressure changes with measured changes in DPTT. This calculated rate may then be combined with an initial measurement of actual blood pressure to provide a calibration model for the relationship between blood pressure and DPTT.

As one example, consider a situation in which sensors are placed on a patient's finger and shoulder to measure an arterial path in the patient's arm. If the elbow is kept straight, then the average height of the differential arterial path is half the height to which the finger is raised or lowered. Let T₁ be the DPTT measured with the arm in a first configuration in which the finger is a distance h₁ above the heart, and let T₂ be the DPTT measured with the arm in a second configuration in which the finger is a distance h₂ above the heart. In some embodiments, h₁ and/or h₂ could be negative if the finger is below the heart, or zero if the finger is level with the heart. The hydrostatic pressure difference between the two measurements at the finger is given by

ΔP_{finger}=ρg(h₂−h₁),

where ρ is the density of blood and g is the gravitational constant. Since the average height difference of the differential arterial path is half the finger height difference, the average pressure change along the differential arterial path is given by

ΔP_{DAP}=ρg(h₂−h₁)/2.

Incorporating the nonlinear DPTT/blood pressure relationship p=a+b·ln(T) into this equation results in

ΔP_{DAP}=b*[ln(T₂)−ln(T₁)]=b*ln(T₂/T₁),

or

b=ΔP_{DAP}/ln(T₂/T₁).

An initial measurement of blood pressure may provide the coefficient a, thus allowing blood pressure to be derived from the relationship p=a+b·ln(T). This relationship is derived from the Moens-Kortewig-Hughes (MKH) equation. The MKH equation is derived by combining the Moens-Kortewig equation for the speed of propagation of a pressure wave in an elastic tube,

V=\sqrt{tE/ρd},

with the Hughes equation for the observed modulus of elasticity of canine aortic tissue,

E=E₀e^{λP},

which is discussed in more detail in Hughes et al., “Measurement of Young's Modulus of Elasticity of the Canine Aorta with Ultrasound,” Ultrasonic Imaging v.1, pp. 356-367, 1979, which is incorporated by reference herein in its entirety. The derivation of the MKH equation is discussed in more detail in Geddes, Handbook of Blood Pressure Measurement, (Humana Press, 1991), which is also incorporated by reference herein in its entirety. The derivation shows that the degree to which the arterial path stiffens with increasing pressure determines the degree to which pulse wave velocity increases with pressure.

In the example discussed above, the differential arterial path is from the shoulder to the finger, and thus primarily includes peripheral arteries rather than aortic arteries. Since peripheral arteries do not necessarily behave in the same manner as aortic tissues (i.e. with exponentially increasing stiffness with increasing pressure), conventional derivations of transit time-blood pressure relationships with parameters measured from aortic tissues may not accurately reflect the relationship of blood pressure to pulse transit time in peripheral arteries. Moreover, different patients may exhibit different blood vessel behavior. The gravity-based calibration method discussed here measures parameters that are specific to the measured arteries of particular patients, and provides an individualized measure of patient arterial stiffening with pressure, thus providing more accurate tracking of pressure changes for each particular patient. For example, some people (e.g., well-conditioned athletes) may have peripheral arteries that are so elastic that arterial stiffening is greatly reduced or even nonexistent as blood pressure increases. The gravity-based calibration method allows the detection of this type of behavior, and allows the system or the clinician to modify operating procedures accordingly. Another benefit of this calibration method is that it provides an immediate indication of the sensitivity of the measuring device for the specific patient being measured. Thus, if for some reason a particular patient's physiology would make blood pressure tracking more difficult, a measure of the patient's physiology can be immediately given to the clinician and/or to the operating software. The clinician and/or operating software may then make changes as appropriate. For example, if an initial calibration indicates that pressure changes yield smaller-than-normal DPTT changes for a particular patient, cuff-based recalibration intervals may be made more frequently.

FIG. 5 is a flow diagram of illustrative steps involved in a gravity-based calibration technique 500. These steps may be performed by the monitoring system 10 described in FIGS. 1-2. At step 502, the monitoring system 10 first determines the differential pulse transit time DPTT1 with the patient in a first position. For example, if the sensor units 12 and 13 (FIG. 1) are placed on one of the patient's arms, at the shoulder and at a finger, respectively, the monitoring system 10 may determine DPTT1 with the patient's arm lying flat. Note that in any of the techniques described herein that are illustrated with reference to two sensors (such as sensor units 12 and 13), three or more sensors may be used to average, filter or otherwise process the inputs or results. At step 504, the monitoring system 10 determines the differential pulse transit time DPTT2 with the patient in a second position. Referring to the example given above, the second position may have the patient's arm and finger raised or lowered with respect to the heart. Once DPTT1 and DPTT2 have been measured, the monitoring system 10 may determine the parameter b, as described above, at step 506. At step 508, if necessary, the monitoring system 10 may also determine the parameter a. For example, a may be determined from the determined b parameter and an additional measured blood pressure value. In other embodiments, the monitoring system 10 may determine the parameter a independently of the gravity-based calibration technique 500. Once values for a and b have been determined, the monitoring system 10 may measure differential pulse transit time and determine blood pressure based on the measured differential pulse transit time, a, and b at step 510. The monitoring system 10 may then determine if a recalibration is needed at step 512, for example, according to a pre-determined schedule or to a dynamic determination. In an embodiment, the monitoring system 10 may determine that a recalibration is needed if a blood pressure change has been detected, if a recalibration has been scheduled, or if a patient parameter such as compliance has changed. Optionally, the recalibration may occur in response to an external input (e.g. from a clinician). If a recalibration is not needed, the monitoring system 10 then proceeds back to step 510 and continues to determine differential pulse transit time and blood pressure. If a recalibration is needed, the monitoring system 10 may determine if a, b, or both parameters need to be recalibrated. If only a needs to be recalibrated, then the monitoring system 10 proceeds to step 508, where a is determined again. If b, or both a and b, need to be recalibrated, then the monitoring system 10 proceeds to step 502.

In other embodiments, other gravity-based calibration techniques may be used to determine the relationship of DPTT and blood pressure. For example, instead of measuring differential pulse transit times with the patient in different positions, the actual patient blood pressures may be measured at each of the different positions. The measured blood pressures may then be used to determine the relationship of DPTT to blood pressure.

Yet another calibration method is a respiration-based calibration technique. Systolic and diastolic blood pressures vary with the respiration cycle, typically about 10-15 mm Hg, due to the pressure changes in the thoracic cavity required to expand and compress the lungs. While respiration cycle and volume are uneven with unaided patients, patients on a ventilator (such as anesthetized patients undergoing surgery) are on a regular respiratory cycle of known volume or pressure changes. Data from the ventilator may be used to correlate the respiratory cycle and thoracic pressure changes with other known or measurable physiological data, such as height, weight, gender, lung volume, resting pulse rate, resting blood pressure, or any other suitable data, to determine the respiratory variation in blood pressure for a given patient. In particular, ventilator respiration data may be used in a respiration-based calibration technique.

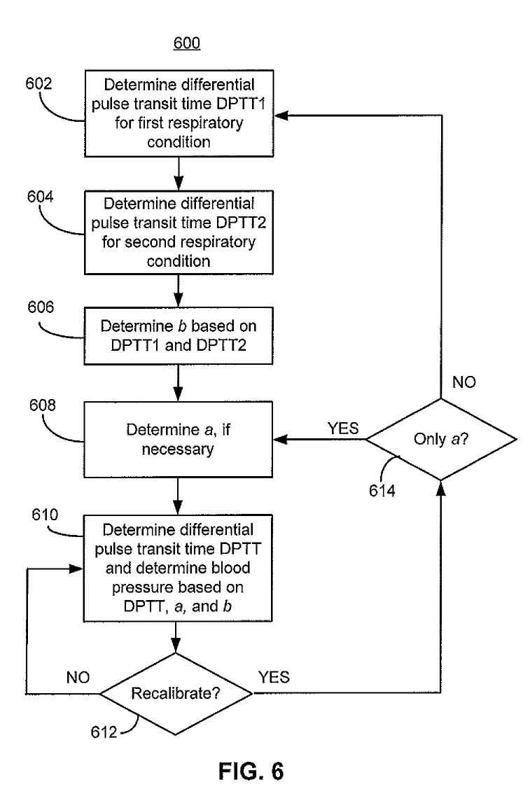

FIG. 6 is a flow diagram of illustrative steps involved in a respiration-based calibration technique 600, which may be performed by monitoring system 10 (FIGS. 1-2). At step 602, the monitoring system 10 may determine a differential pulse transit time DPTT1 for a patient when the patient is in a first respiratory condition. Patient respiratory conditions may be controlled via a device such as a ventilator, which provide the patient with a regular respiratory cycle of known volume or pressure changes. At step 604, the monitoring system 10 may determine a differential pulse transit time DPTT2 for the patient when the patient is in a second respiratory condition. For example, the patient may have mostly evacuated lungs in the first respiratory condition and may have mostly filled lungs in the second respiratory condition. Once DPTT1 and DPTT2 have been determined, monitoring system 10 may use these differential pulse transit times, along with associated respiratory parameters, to determine the parameter b in the DPTT-blood pressure relationship at step 606. In an embodiment, the monitoring system 10 may determine the parameter b with the following equation:

b=ΔP/ln(T₂/T₁),

where the ΔP represents the change in pressure between the first respiratory condition and the second respiratory condition.

At step 608, if necessary, the monitoring system 10 may also determine the parameter a. For example, a may be determined from the determined b parameter and an additional measured blood pressure value. In other embodiments, the monitoring system 10 may determine the parameter a independently of the respiration-based calibration technique 600. Once values for a and b have been determined, the monitoring system 10 may then determine a DPTT of the patient and determine blood pressure from the DPTT and the parameters a and b (step 610). The monitoring system 10 may then determine if a recalibration is needed at step 612, for example according to a pre-determined schedule or to a dynamic determination. In an embodiment, the monitoring system 10 may determine that a recalibration is needed if a blood pressure change has been detected, if a recalibration has been scheduled, or if a patient parameter such as compliance has changed. Optionally, the recalibration may occur in response to an external input (e.g. from a clinician). If a recalibration is not needed, the monitoring system 10 then proceeds back to step 610 and continues to determine differential pulse transit time and blood pressure. If a recalibration is needed, the monitoring system 10 may determine if a, b, or both parameters need to be recalibrated. If only a needs to be recalibrated, then the monitoring system 10 proceeds to step 608, where a is determined again. If b or both a and b need to be recalibrated, then the monitoring system 10 proceeds to step 602.

While the individual calibration techniques described above provide reasonably accurate DPTT-blood pressure relationships for measuring blood pressure, a calibration process that involves multiple calibration techniques may provide even better accuracy for blood pressure determinations, as well as allowing data collected from different techniques to be compared to each other, thus providing better error correction capabilities. For example, the inter-patient calibration technique 300 (FIG. 3) may be able to provide an initial DPTT-blood pressure model quickly, because it is based on parameters that have been previously determined from other patients (e.g., c₁ and c₂). However, the DPTT-blood pressure model from inter-patient calibration technique 300 may become more inaccurate as time passes, because of changes in the patient. In contrast, the intra-patient multi-point calibration technique 400 allows for the refinement of the DPTT-blood pressure model as patient parameters change. However, technique 400 (FIG. 4) may need an initial calibration point or model (e.g., step 402). By combining techniques 300 and 400, possibly in a serial fashion, a DPTT-blood pressure model can be generated quickly and updated continuously for high accuracy. Similarly, the gravity-based calibration technique 500 (FIG. 5) and/or the respiration-based calibration technique 600 (FIG. 6) may be incorporated or combined into the blood pressure/DPTT measurement process to improve the accuracy of measured parameters, such as b. Additional examples of calibration models that may be used with the techniques disclosed herein are described in McCombie et al., “Adaptive hydrostatic blood pressure calibration,” Proceedings of the 29th Annual International Conference of the IEEE EMBS, 2007, and McCombie et al., “Motion based adaptive calibration of pulse transit time measurements to arterial blood pressure for an autonomous, wearable blood pressure monitor,” Proceedings of the 30th Annual International Conference of the IEEE EMBS, 2008, both of which are incorporated by reference in their entirety herein.

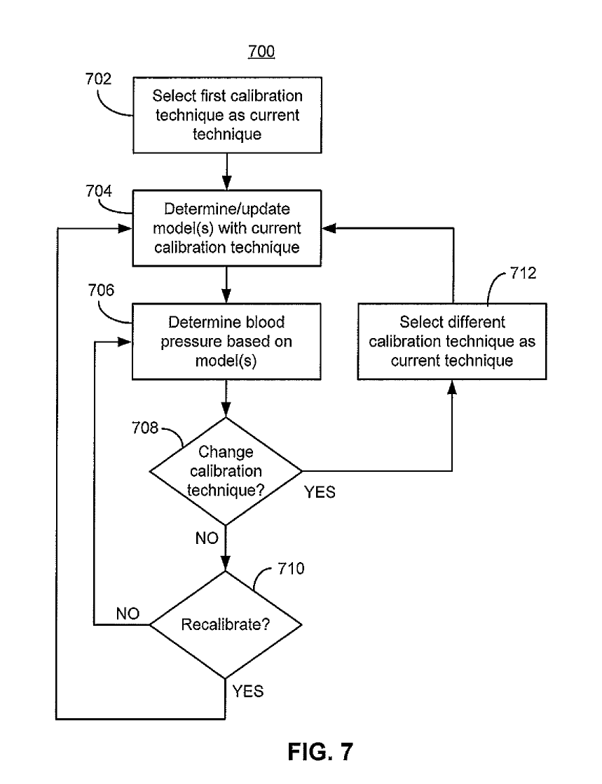

FIG. 7 is a flow diagram of illustrative steps involved in a DPTT-blood pressure model serial calibration technique 700, which may be performed by monitoring system 10 (FIGS. 1-2). At step 702, the monitoring system 10 may select a first calibration technique as a current technique. In some embodiments, this selection may be based upon a determination of how quickly a calibration technique can provide a suitable DPTT-blood pressure model. This determination may be pre-set or performed by the monitoring system 10. At step 704, the monitoring system 10 may determine one or more DPTT-blood pressure models with the current technique. In some embodiments, although the DPTT-blood pressure models are determined and/or updated with a particular calibration technique, the model data may also be stored for use with other calibration techniques, as described below. After determination of the model(s), the monitoring system 10 may proceed to step 706 to determine blood pressure with the DPTT-blood pressure model(s); for example, by calculating blood pressure from one or more measured DPTTs. At step 708, the monitoring system 10 may check to see if it should change calibration techniques. This check may be based on a pre-set schedule (e.g., changing to a different technique after a certain amount of time has passed), or may be based upon a dynamic determination by the monitoring system 10 or by an operator. For example, the monitoring system 10 or the operator may decide that the current technique is becoming too inaccurate, and that a new calibration technique should be used. In some embodiments, new patient data may become available, and the monitoring system 10 or the operator may decide to use a different calibration technique that can utilize the new patient data.

If it is determined that the current calibration technique should be changed, the monitoring system 10 may proceed to step 712, where the monitoring system 10 selects a different calibration technique as a new current technique. This selection may be based upon characteristics of the calibration techniques, such as speed, as well as the data available. For example, if respiratory data is available for the patient, the monitoring system 10 may select a calibration technique that takes advantage of respiratory data (e.g., technique 600 of FIG. 6). The monitoring system 10 may also select a different calibration technique based on the amount of model data already accumulated. As discussed above, in relation to step 704, the model data obtained from a particular calibration technique may be used for other calibration techniques. For example, the inter-patient empirical data calibration technique described in FIG. 3 only requires a single calibration point to generate a DPTT-blood pressure model. Other, calibration techniques may require multiple model calibration points to provide more accurate models. Thus, in one embodiment, the serial calibration technique 700 may first begin with the inter-patient empirical data calibration technique (or any other technique capable of quickly generating an initial model). After sufficient model calibration data has been collected with the first technique, the monitoring system 10 may select a more accurate calibration technique that requires more model calibration data for accuracy. In this way, the monitoring system 10 may transition between calibration techniques that are less accurate but require fewer calibration points and more accurate calibration techniques that require more calibration points. After selecting the new current technique, the monitoring system 10 may proceed to step 704, where it updates the current DPTT-blood pressure model(s) and/or generates one or more entirely new models. The decision of whether the current model is updated or replaced may be based on changes in patient parameters (e.g., compliance) or how closely the parameters generated by the new calibration technique match the current model.

If, at step 708, the monitoring system 10 determines that the calibration technique does not need to be changed, then the monitoring system 10 may proceed to step 710, where it determines if a recalibration should be performed. The monitoring system 10 may determine if a recalibration should be performed based on a pre-set schedule (e.g., recalibrate every ten minutes) or based on a dynamic determination (e.g., based on a change event in which a patient parameter change exceeds some preset or dynamically-determined threshold). If the monitoring system 10 determines that a recalibration should be performed, the monitoring system 10 may proceed to step 704 and perform a recalibration with the current calibration technique and to update the current model or other models with the recalibration data. If the monitoring system 10 determines that a recalibration is not necessary, the monitoring system 10 may revert to step 706.

In some embodiments, a multi-calibration technique may be used, with multiple calibration techniques occurring simultaneously and/or substantially simultaneously (e.g., in parallel). For example, the intra-patient multi-point calibration technique 400 may be performed simultaneously with the respiration-based calibration technique 600. Each of the multiple calibration techniques will result in an associated set of model parameters, and the multiple associated sets of model parameters may be combined by, for example, a weighted average of the sets. The weighted model parameter averages may be more accurate than model parameters from individual calibration techniques. The weighting may be determined by a preset or stored schema, or may be dynamically determined by the monitoring system 10 (FIGS. 1-2) and/or an operator. For example, a dynamic determination may be based on changes in patient parameters (e.g. compliance) or changes in data availability (e.g., respiration data becomes available or unavailable). In some embodiments, one or more sets of particular model parameters may be discarded, based on a determination that may be performed by the monitoring system 10 (FIGS. 1-2) and/or an operator.

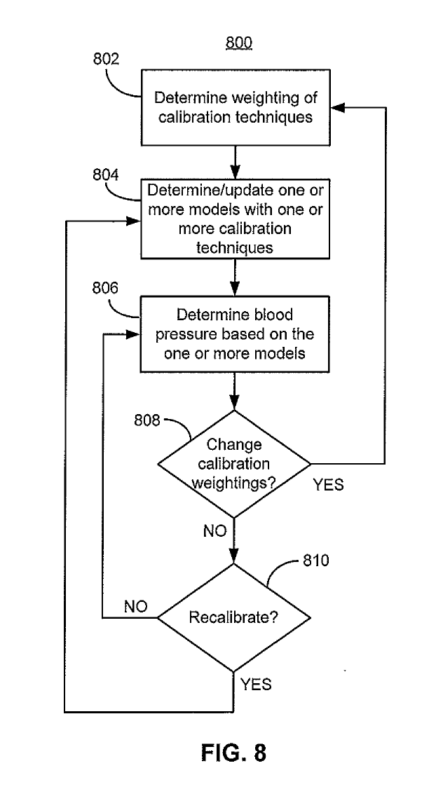

FIG. 8 is a flow diagram of illustrative steps involved in a multi-calibration technique 800. In some embodiments, multi-calibration technique 800 may be performed by monitoring system 10 (FIGS. 1-2). At step 802, the monitoring system 10 may determine one or more calibration techniques to use, such as the calibration techniques described above in relation to FIGS. 3-6, and determine a weighting associated with each calibration technique. These determinations may be based on patient parameters, characteristics of the calibration techniques, and the available data. The weighting may be performed by a weighted averaging process, a distribution process, or any other weighted combination process. The weighting may be applied at different points during the multi-calibration technique 800 (FIG. 8). In an embodiment, the contribution of each calibration technique to a particular variable in a DPTT-blood pressure model may be weighted. For example, an inter-patient empirical calibration technique (e.g., as illustrated in FIG. 3) may be weighted to contribute more to the a variable in the model than a gravity-based calibration technique (e.g., as illustrated in FIG. 5), but the gravity-based calibration technique may be weighted to contribute more to the b variable in the model than the inter-patient technique. In some embodiments, the contribution of each calibration technique to the entire DPTT-blood pressure model may be weighted. In these embodiments, a particular calibration technique may be weighted to contribute equally to all of the variables in the model, but different calibration techniques may be weighted to contribute differently to the model. For example, an inter-patient calibration technique may be weighted to contribute half as much as a gravity-based calibration technique to all the variables in the model. In this example, each model variable would obtain approximately 33% of its value from the inter-patient calibration technique and approximately 66% from the gravity-based calibration technique. In certain embodiments, each calibration technique may be used to develop a separate DPTT-blood pressure model. Each of these models may be used to determine a separate blood pressure, and then the separate blood pressures may be combined according to the weighting to generate a final blood pressure. In an embodiment, the weighting of the calibration techniques and/or the sets of model parameters may be based on an indication of accuracy of the calibration technique and/or the model. These indications of accuracy may be based on a subject's blood pressure, the number of data points, an analysis of the data points, the type of model, the type of calibration technique, or any other indication of accuracy. Any other method of weighting and/or distributing the calibration techniques and/or generated models may be used.

Once the calibration techniques have been selected and their weights assigned at step 802, the monitoring system 10 may proceed to step 804, and determine and/or update one or more DPTT-blood pressure models with the one or more calibration techniques. With the updated DPTT-blood pressure model(s), the monitoring system 10 may then determine one or more blood pressure values by, for example, measuring DPTT and calculating blood pressure based on the one or more models and determined weightings. The monitoring system 10 may then determine if the weightings associated with the calibration techniques are to be changed at step 808. The determination may be based on a pre-set schedule (e.g., every ten minutes or every hour on the hour) or may be a dynamic determination by the monitoring system 10 and/or an operator. For example, additional patient respiration data may become available, or a calibration technique may have just been updated with new calibration data. In such embodiments, the calibration technique weightings may be updated to take advantage of the new, more accurate data. Alternately, if data becomes unavailable, calibration techniques that use the now-unavailable data may be weighted less than other techniques. If the monitoring system 10 determines that the calibration weightings should be changed, the monitoring system 10 proceeds to step 802, where the weights associated with the current calibration techniques are re-determined. In some embodiments, new calibration techniques may be added, or current calibration techniques may be removed. Optionally, the removal of a calibration technique may be accomplished simply by assigning that calibration technique a zero weighting.

Alternately, if calibration weightings do not need to be changed at step 808, the monitoring system 10 proceeds to step 810, where the monitoring system 10 determines if a recalibration should be performed. This determination may be based on a pre-set or stored schedule or based on a dynamic determination. If the monitoring system 10 determines that a recalibration should be performed, then the monitoring system 10 proceeds to step 804, where recalibration occurs and the one or more models are updated with the recalibrated values. Recalibration may occur for only some of the calibration techniques, or for all of the recalibration techniques. In some embodiments, recalibration for each calibration technique is governed individually. For example, the monitoring system 10 and/or an operator may examine each calibration technique separately to determine if it should be recalibrated. This determination may be based on a pre-set or pre-determined schedule, patient parameters and parameter changes, and/or newly available or unavailable data. If the monitoring system 10 decides that a recalibration should not be performed, the monitoring system 10 may proceed to step 806, where the monitoring system 10 continues to determine blood pressure based on the one or more current models.

Variations, modifications, and other implementations of what is described may be employed without departing from the spirit and scope of the disclosure. More specifically, any of the methods, systems and device features described above or incorporated by reference may be combined with any other suitable methods, systems, or device features disclosed herein or incorporated by reference, and is within the scope of the disclosure. The systems and methods may be embodied in other specific forms without departing from the spirit or essential characteristics thereof. The foregoing embodiments are therefore to be considered in all respect illustrative, rather than limiting. The teachings of all references cited herein are hereby incorporated by reference in their entirety.

1. A method for measuring blood pressure of a subject, comprising:
determining, with a processor executing a first calibration technique, first calibration data for use in determining blood pressure;
determining, with a processor executing a second calibration technique, second calibration data for use in determining blood pressure, wherein the second calibration technique is different from the first calibration technique;
measuring a differential pulse transit time; and
determining, with a processor, the blood pressure of the subject based at least in part on the differential pulse transit time and at least one of the first calibration data and the second calibration data.

2. The method of claim 1, wherein the first calibration technique is based at least in part on at least one of inter-patient empirical data, intra-patient empirical data, gravity-based calibration data, and respiration-based calibration data.

3. The method of claim 1, wherein the first calibration data and the second calibration data are determined substantially simultaneously.

4. The method of claim 1, wherein the first calibration data is determined substantially before the second calibration data is determined.

5. The method of claim 1, wherein the blood pressure is determined based at least in part on a weighted combination of the first calibration data and the second calibration data.

6. The method of claim 5, wherein the weighting of the combination of the first calibration data and the second calibration data is based at least in part on an indication of accuracy.

7. The method of claim 1, wherein the first calibration data includes a plurality of calibration points, wherein the plurality of calibration points are weighted based at least in part on when the calibration points were determined, and wherein the blood pressure is determined based at least in part on the weighted calibration points.

8. The method of claim 1, further comprising:
determining, with a processor, if at least one of the first calibration data and the second calibration data include outlier data; and
in response to determining that outlier data is included, determining, with a processor, if the outlier data should be removed.

9. A method for measuring blood pressure of a subject, comprising:
determining, with a processor executing a calibration technique, first calibration data;
measuring a first differential pulse transit time;
determining, with a processor, the blood pressure of the subject based at least in part on the first differential pulse transit time and the first calibration data;
determining that a change event occurred;
determining, with a processor executing a calibration technique, second calibration data based on at least two calibration data points taken after the change event occurred;
measuring a second differential pulse transit time after the change event occurred; and
determining, with a processor, the blood pressure of the subject based at least in part on the second differential pulse transit time and the second calibration data after the change event occurred.

10. The method of claim 9, wherein the change event is a change in compliance.

11. The method of claim 9, further comprising storing, in a memory, for later use, at least one of the first calibration data, the first differential pulse transit time, the second calibration data, the second differential pulse transit time, and the blood pressure.

12. The method of claim 9, wherein the calibration technique executed before the change event is different from the calibration technique executed after the change event.

13. The method of claim 9, wherein the second calibration data is further based on calibration data previously stored in a memory for later use.

14. A system for measuring blood pressure of a subject, comprising:
at least two sensors; and
a processor configured to:
execute a first calibration technique to determine first calibration data for use in determining blood pressure;
execute a second calibration technique to determine second calibration data for use in determining blood pressure, wherein the second calibration technique is different from the first calibration technique;
measure a differential pulse transit time based at least in part on data received from the at least two sensors; and
determine the blood pressure of the subject based at least in part on the differential pulse transit time and at least one of the first calibration data and the second calibration data.

15. The system of claim 14, wherein the first calibration technique is based at least in part on at least one of inter-patient empirical data, intra-patient empirical data, gravity-based calibration data, and respiration-based calibration data.

16. The system of claim 14, wherein the processor is configured to determine the first calibration data and the second calibration data substantially simultaneously.

17. The system of claim 14, wherein the processor is configured to determine the first calibration data substantially before determining the second calibration data.

18. The system of claim 14, wherein the processor is configured to determine blood pressure based at least in part on a weighted combination of the first calibration data and the second calibration data.

19. The system of claim 18, wherein the weighting of the combination of the first calibration data and the second calibration data is based at least in part on an indication of accuracy.

20. The system of claim 14, wherein the first calibration data includes a plurality of calibration points, wherein the calibration points are weighted based at least in part on when the calibration points were determined, and wherein the processor is configured to determine blood pressure based at least in part on the weighted calibration points.

21. The system of claim 14, wherein the processor is configured to:
determine if at least one of the first calibration data and the second calibration data include outlier data; and
in response to determining that outlier data is included, determine if the outlier data should be removed.

22. A system for measuring blood pressure of a subject, comprising:
at least two sensors; and
a processor configured to:
execute a calibration technique to determine first calibration data;
measure a first differential pulse transit time based at least in part on data received from the at least two sensors;
determine the blood pressure of the subject based at least in part on the first differential pulse transit time and the first calibration data;
determine that a change event occurred;
execute a calibration technique to determine second calibration data based on at least two calibration data points taken after the change event occurred;
measure a second differential pulse transit time based at least in part on data received from the at least two sensors after the change event occurred; and
determine the blood pressure of the subject based at least in part on the second differential pulse transit time and the second calibration data after the change event occurred.

23. The system of claim 22, wherein the change event is a change in compliance.

24. The system of claim 22, further comprising a memory, and wherein the processor is configured to store, in the memory, for later use, at least one of the first calibration data, the first differential pulse transit time, the second calibration data, the second differential pulse transit time, and the blood pressure.

25. The system of claim 22, wherein the calibration technique executed before the change event is different from the calibration technique executed after the change event.

26. The system of claim 22, further comprising a memory, wherein the second calibration data is further based on calibration data previously stored in the memory for later use.
